(19) United States
(12) Patent Application Publication
(10) Pub. No.: US 2004/0172106 A1
(43) Pub. Date: Sep. 2, 2004

(54) Apparatus for ablation with a laser beam

(76) Inventor: Satoshi Imaizumi, Hoi-gun (JP)

Correspondence Address: RADER FISHMAN & GRAUER PLLC, LION BUILDING, 1233 20TH STREET N.W., SUITE 501, WASHINGTON, DC, 20036, US

(21) Appl. No.: 10/689,095

(22) Filed: Oct. 21, 2003

(30) Foreign Application Priority Data
Oct. 24, 2002 (JP) JP2002-310261

(51) Int. Cl. A61N 1/00

(52) U.S. Cl. 607/89

(57) ABSTRACT

An ablation apparatus capable of obtaining intensity distribution in a concave shape at an irradiation surface even when a laser beam goes through a small opening of an aperture to be formed into a small spot. In the apparatus, a laser light source emits the beam which effects ablation to the object, an irradiation optical system directs and irradiates the beam onto an irradiation surface of the object, an aperture in the system has an opening, a convex lens in the system once collects the beam passed through the opening and directs the beam onto the surface at a defocus position, and an aspherical optical element in the system makes intensity distribution of the beam after passing through the opening to be convex, wherein an aspherical shape of the element is curved where a radius of curvature at a local surface is reduced toward a periphery from an optical axis.

### BACKGROUND OF THE INVENTION

1. Field of the Invention

The present invention relates to an ablation apparatus for ablating an object such as eye tissue with a laser beam.

2. Description of Related Art

Conventionally, there is known an apparatus which performs ablation by irradiating an excimer laser beam onto corneal tissue for correcting a refractive error of an eye or removing an affected part at a corneal surface. According to one method of laser beam irradiation, this kind of apparatus performs irradiation by means of two-dimensionally moving (scanning) the laser beam of a small spot of about 1 mm on the cornea. At this time, if intensity distribution of the spot laser beam at an irradiation surface is made in a convex shape and the laser beam is irradiated while being interlocked at an appropriate ratio, an ablation surface may be made smooth.

Incidentally, when the laser beam is passed through a small opening of an aperture to be formed into a small spot, influence of diffraction due to the aperture (opening) grows, and intensity distribution of the laser beam at the irradiation surface becomes a concave shape. A conventional technique for reducing a diffraction component produced by the aperture (opening) provides a method of using an apodization filter (a density distribution filter in which transmittance is high in a central portion within an effective diameter and is reduced toward a peripheral portion). However, in a case where the effective diameter of the opening of the aperture is as small as 3 mm, there is a problem that a membrane with density distribution is hard to constitute. Further, in a case where a laser output is required to be 100 mJ or higher such as in corneal ablation, there is a problem in durability of a coat membrane.

### SUMMARY OF THE INVENTION

An object of the invention is to overcome the problems described above and to provide an ablation apparatus capable of obtaining intensity distribution in a concave shape at an irradiation surface even when a laser beam is passed through a small opening of an aperture to be formed into a small spot.

To achieve the objects and in accordance with the purpose of the present invention, an apparatus for ablation with a laser beam has a laser light source for emitting the laser beam which effects ablation to the object, an irradiation optical system for directing and irradiating the laser beam emitted from the laser light source onto an irradiation surface of the object, an aperture, arranged in the irradiation optical system, having an opening, a convex lens arranged in the irradiation optical system for once collecting the laser beam passed through the opening of the aperture and then directing the laser beam onto the irradiation surface at a defocus position, and an aspherical optical element arranged in the irradiation optical system for making intensity distribution of the laser beam after passing through the opening of the aperture to be intensity distribution in a convex shape, wherein an aspherical shape of the aspherical optical element is a curved shape where a radius of curvature at a local surface is reduced toward a peripheral portion from an optical axis.

Additional objects and advantages of the invention are set forth in the description which follows, are obvious from the description, or may be learned by practicing the invention. The objects and advantages of the invention may be realized and attained by the ablation apparatus using the laser beam in the claims.

### BRIEF DESCRIPTION OF THE DRAWINGS

The accompanying drawings, which are incorporated in and constitute a part of this specification, illustrate embodiments of the present invention and, together with the description, serve to explain the objects, advantages and principles of the invention. In the drawings,

FIG. 1 is a view showing a schematic configuration of an irradiation optical system and a control system in an example where the present invention is applied to an ablation apparatus for corneal surgery;

FIG. 2 is a view showing a typical shape of an excimer laser beam;

FIGS. 3A and 3B are views showing a schematic configuration of a dividing aperture plate and a shutter device;

FIG. 4 is a view illustrating irradiation performed by interlocking a laser beam of a small spot;

FIG. 5 is a schematic view showing the irradiation optical system in the ablation apparatus consistent with the present invention;

FIG. 6 is a schematic view showing the irradiation optical system in a case where an aspherical optical element is not arranged as different from FIG. 5;

FIG. 7 is a view where the irradiation optical system in FIG. 5 is marked with an opening diameter φ of an aperture, a focal length f of a convex lens, a distance d from the aspherical optical element to the convex lens, and a defocus amount L from a focal point of the convex lens to an irradiation surface;

FIG. 8 is a view showing a result of simulation of intensity distribution at the irradiation surface;

FIG. 9 is a view showing another result of simulation of the intensity distribution at the irradiation surface;

FIG. 10 is a view showing still another result of simulation of the intensity distribution at the irradiation surface;

FIG. 11 is a view showing still another result of simulation of the intensity distribution at the irradiation surface;

FIG. 12 is a view showing still another result of simulation of the intensity distribution at the irradiation surface;

FIG. 13 is a view showing a relation between an exponential coefficient “a” when an aspherical shape is expressed by Formula 1 giving an exponential function and the focal length f;

FIG. 14 is a view showing the aspherical shape where intensity distribution in a convex shape is obtained within a range of the focal length f=50 to 500 (mm);

FIG. 15 is a view showing a range of the opening diameter φ of the aperture when the aspherical shape is expressed by Formula 1 giving the exponential function; and

FIG. 16 is a view showing a range of the distance d, in which intensity distribution in a preferable convex shape is obtained, with respect to the focal length f=50 to 500 (mm).

### DETAILED DESCRIPTION OF THE PREFERRED EMBODIMENTS

A detailed description of one preferred embodiment of an apparatus for ablation with a laser beam embodied by the present invention is provided below with reference to the accompanying drawings. FIG. 5 is a schematic view showing an irradiation optical system in an ablation apparatus consistent with the present invention.

Reference numeral 201 is a laser beam emitted from a laser light source, 203 is an aspherical optical element, 205 is an aperture having a small opening, 207 is a convex lens, and 209 is an irradiation surface. For the laser beam 201 which effects ablation, an excimer laser beam with a wavelength of 193 nm may be preferably used. For the laser beam 201 entering the aspherical optical element 203, assume that intensity distribution in a beam cross section perpendicular to an irradiation optical axis L0 is made uniform by uniforming means not illustrated. The aspherical optical element 203 has a flat shape on the laser light source side and an aspherical shape on the irradiation surface 209 side, and the aspherical shape is a curved shape where a radius of curvature of its surface is reduced toward the periphery from the optical axis L0 (a center of the opening of the aperture 205). In addition, synthetic fused quartz may be preferably used as a material for the aspherical optical element 203. Besides, in FIG. 5, the aspherical optical element 203 is arranged on the laser light source side with respect to the aperture 205. However, it may be arranged on the irradiation surface 209 side. Further, the aspherical optical element 203 is preferably arranged in the vicinity of the aperture 205.

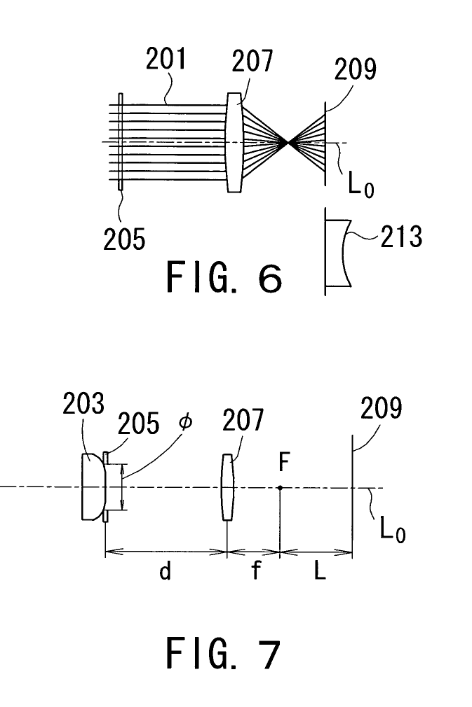

The laser beam 201 passed through the opening of the aperture 205 is once collected by the convex lens 207, and then irradiated onto the irradiation surface 209. Here will be considered a case where the aspherical optical element 203 is not arranged. In this case, ray intervals of the laser beam 201 are equal, and the intensity distribution at the irradiation surface 209 has a concave shape 213 by the influence of diffraction due to the aperture 205 (opening) (see FIG. 6). On the other hand, in a case where the aspherical optical element 203 is arranged, the ray intervals in a peripheral portion of the laser beam 201 are lengthened, so that the intensity distribution at the irradiation surface 209 is reduced toward the periphery (see FIG. 5). Therefore, the aspherical shape (curved shape) of the aspherical optical element 203 is determined so as to lengthen the ray intervals in the peripheral portion of the laser beam 201, so that the intensity distribution in a concave shape 213 may be changed into intensity distribution in a convex shape 211.

On determining the aspherical shape (curved shape) an aspherical polynomial expression is generally used. However, the expression has too many parameters to set, which causes complicated operation. Hence, attention was given on a fact that the aspherical shape in which a radius of curvature at a local surface is reduced toward the periphery from the optical axis may be expressed by an exponential function, and it was found that the intensity distribution in a convex shape is obtained by Formula 1 given below. In Formula 1, Y indicates a distance (mm) from the optical axis, and Z indicates a sag amount (mm).

Z=×exp[a×Y⁵]+1 Formula 1:

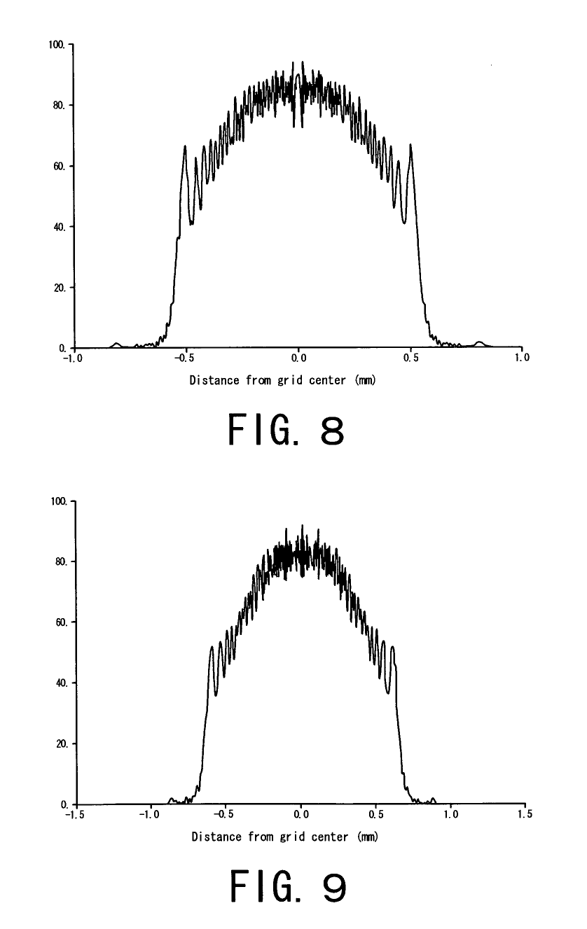

FIGS. 8 to 12 show results of simulation of the intensity distribution at the irradiation surface 209 by using Formula 1. On the simulation, essential conditions are an opening diameter φ of the aperture 205, a focal length f of the convex lens 207, a distance d from the aspherical optical element 203 to the convex lens 207, a defocus amount L from a focal point of the convex lens 207 to the irradiation surface 209 (see FIG. 7), and an exponential coefficient “a” presented in Formula 1.

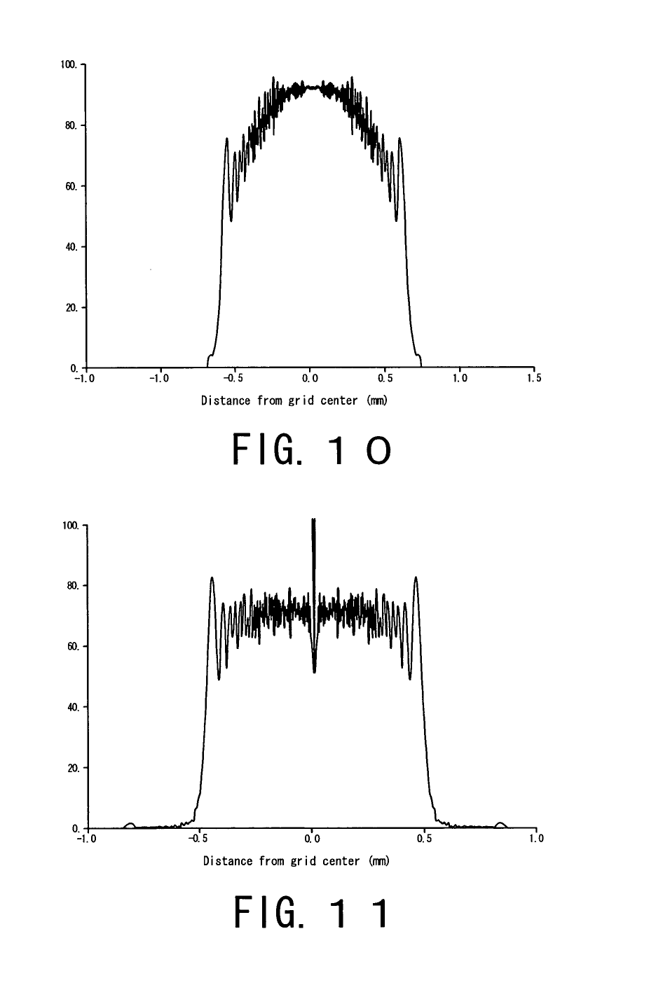

FIG. 8 shows the result in the case of φ=3.2 mm, f=50 mm, d=40 mm, L=15 mm, and a=0.00009. FIG. 9 shows the result in the case of φ=3.2 mm, f=220 mm, d=700 mm, L=75 mm, and a=0.00012. FIG. 10 shows the result in the case of φ=3.2 mm, f=500 mm, d=1800 mm, L=175 mm, and a=0.00009. In either case in FIGS. 8 to 10, the intensity distribution in a convex shape was obtained.

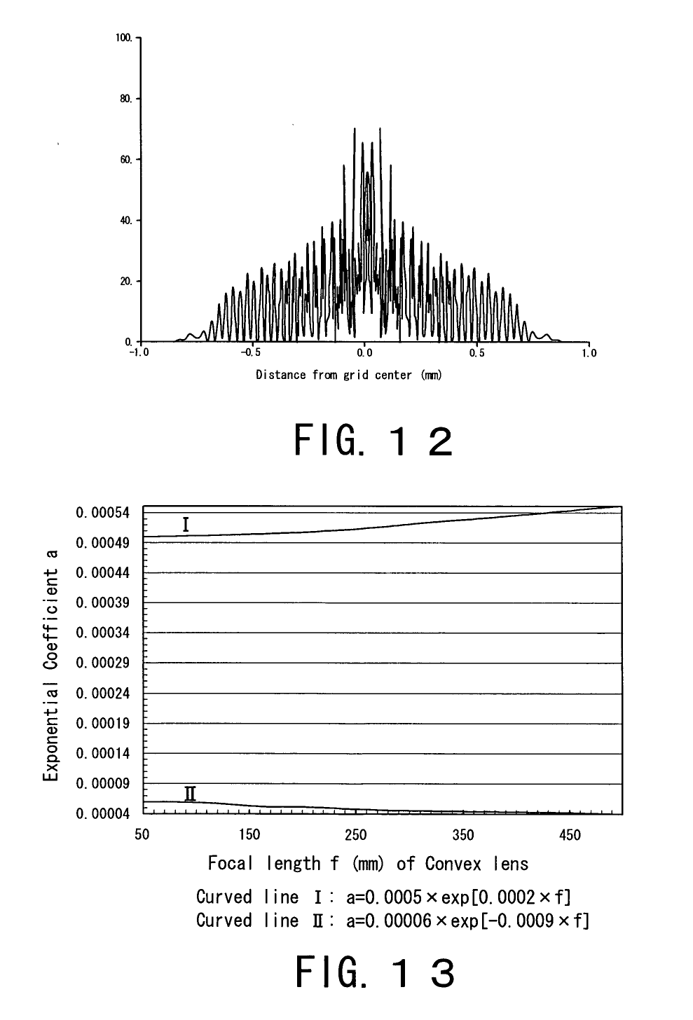

On the other hand, FIG. 11 shows the result where a=0.00002 is provided and the other conditions are the same as those in FIG. 8. FIG. 12 shows the result where a=0.00008 is provided and the other conditions are the same as those in FIG. 8. In the case of FIG. 11, components in edge portions of the intensity distribution become too strong (the influence of the diffraction due to the aperture 205 (opening) still remains). Contrarily, in the case of FIG. 12, the components in the edge portions of the intensity distribution become too weak, and steep components appear in a central portion. Therefore, the distribution in either case cannot be applied as the intensity distribution in a convex shape.

As described above, while variously changing each condition, the intensity distribution at the irradiation surface 209 was simulated to know the aspherical shape (curved shape) of the aspherical optical element 203 in which the intensity distribution in a preferable convex shape is obtained. In this regard, the focal length f of the convex lens 207 was set within a range of 50 to 500 (mm), and a spot diameter (width) of the laser beam 201 used for ablating eye tissue was set within a range of 0.5 to 2.0 (mm) (half breadth of the intensity) at the irradiation surface 209.

FIG. 13 shows a relationship between the exponential coefficient “a” when the aspherical shape (curved shape) is expressed by Formula 1 and the focal length f of the convex lens 207. Within the range of the focal length f=50 to 500 (mm), the exponential coefficient “a” is preferably in a range between Curved line I and Curved line II shown in FIG. 13, which are respectively expressed as follows.

a=0.0005×exp[0.0002×f] Curved line I:

a=0.00006×exp[−0.0009×f] Curved line II:

That is to say, the exponential coefficient “a” is preferably in a range of:

0.00006×exp[−0.0009×f]≦a≦0.0005×exp[0.0002×f].

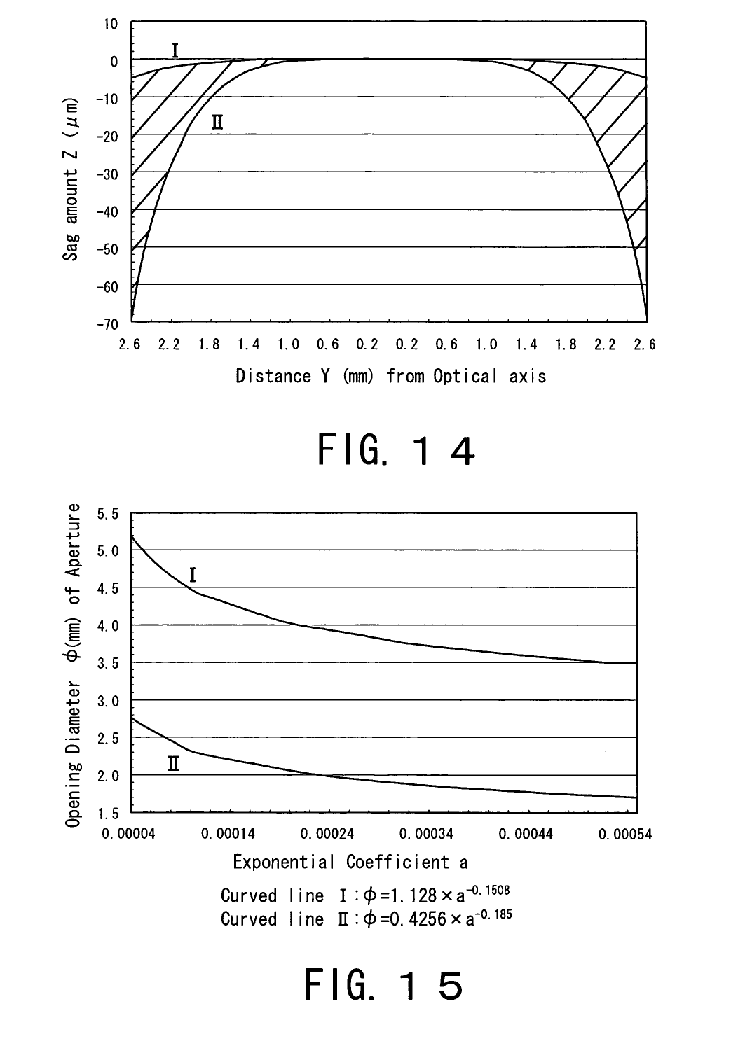

FIG. 14 shows the aspherical shape in which the intensity distribution in a convex shape is obtained within the range of the focal length f=50 to 500 (mm). A preferable curved shape has a curved surface which falls within a range between Curved line I and Curved line II shown in FIG. 14 (a curved surface in which the radius of curvature of its surface is reduced toward the periphery from the optical axis). FIG. 14 gives a cross-sectional view where the curved shape is in a rotationally symmetrical shape having the optical axis as the center. When Formula 1 is used as a method for expressing the aspherical shape (curved shape), based on the results in FIG. 13, the exponential coefficient “a” is preferably in a range of:

0.00004≦a≦0.00055.

Provided below is Formula 2 which is an aspherical polynomial expression of the order 10.

Z=AY⁴+BY⁶+CY⁸+DY¹⁰ Formula 2:

When the aspherical shape (curved shape) is expressed by Formula 2, each of coefficients A, B, C and D is in a range of:

−2.25×10⁻⁵≦A≦−3.01×10⁻⁴

−2.03×10⁻⁵≦B≦−2.80×10⁻⁴

1.87×10⁻⁶≦C≦2.58×10⁻⁵

−9.72×10⁻⁸≦D≦−1.49×10⁻⁶.

Besides, the method given above for expressing the aspherical shape (curved shape) is only an exemplification, and a method of expressing by a truncated binary power function of tangent and the like may be employed.

In addition, FIG. 15 is a view showing a range of the opening diameter of the aperture 205 when the aspherical shape (curved shape) is expressed by Formula 1. With respect to the exponential coefficient “a”, the opening diameter φ is preferably within a range between Curved line I and Curved line II shown in FIG. 15, which are respectively expressed as follows.

φ=1.128×a^{-0.1508} Curved line I:

φ=0.4256×a^{-0.185} Curved line II:

That is to say, the opening diameter is preferably in a range of:

0.4256×a^{-0.185}≦φ≦1.128×a^{-0.1508} (mm).

Further, with respect to the opening diameter φ (mm) within the range shown in FIG. 15 and the focal length f=50 to 500 (mm), the defocus amount L from the focal point of the convex lens 207 to the irradiation surface 209 is preferably within a range of:

0.8×(f/φ)≦L≦2.0×(f/φ) (mm).

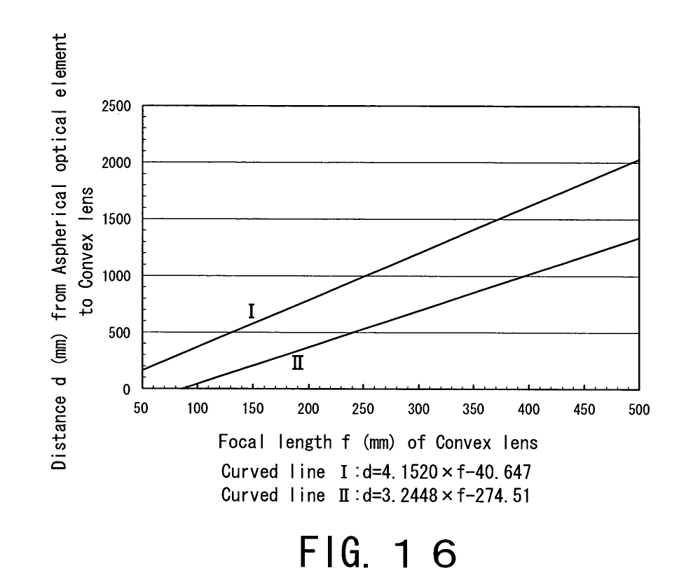

FIG. 16 is a view showing a range of the distance d from the aspherical optical element 203 to the convex lens 207 with respect to the focal length f=50 to 500 (mm), in which range the intensity distribution in a preferable convex shape is obtained. The distance d is preferably within a range between Curved line I and Curved line II shown in FIG. 16, which are respectively expressed as follows.

d=4.1520×f−40.647 Curved line I:

d=3.2448×f−274.51 Curved line II:

That is to say, the distance d is preferably in a range of:

3.2448×f−274.51≦d≦4.1520×f−40.647 (mm).

The above-mentioned φ, L, and d are derived from the result of simulation of the intensity distribution at the irradiation surface 209 by using Formula 1.

A more preferable example will be described with regard to the aspherical shape (curved shape) of the aspherical optical element 203. When φ=3.2 mm, f=220 mm, d=660 mm, and L=78 mm were established in FIG. 7, an aspherical shape where the spot diameter (width) of the laser beam 201 at the irradiation surface 209 satisfies 11.0 mm±0.2 mm in half breadth of the intensity was as follows. As a result of simulating the intensity distribution at the irradiation surface 209, the exponential coefficient “a” in Formula 1 is in a range of:

0.00006≦a≦0.00012.

It is particularly preferable if a=0.00009 is provided. When the aspherical shape of the aspherical optical element 203 is actually manufactured, the exponential function with these conditions is exercised using the aspherical polynomial expression of the order 10 in Formula 2 so that the aspherical shape may be easily manufactured.

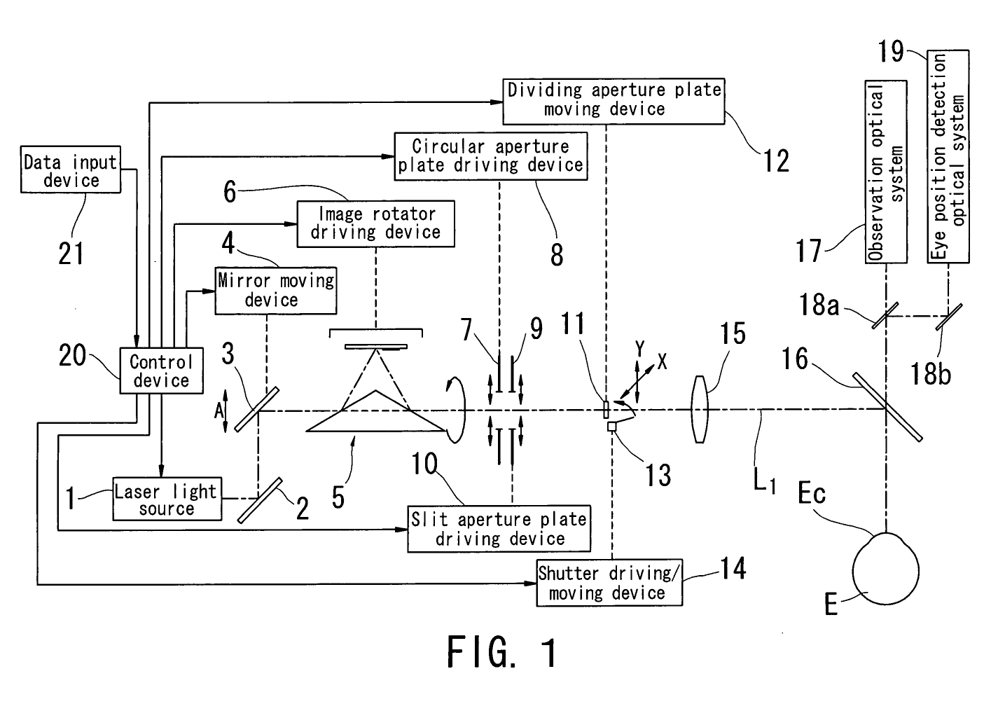

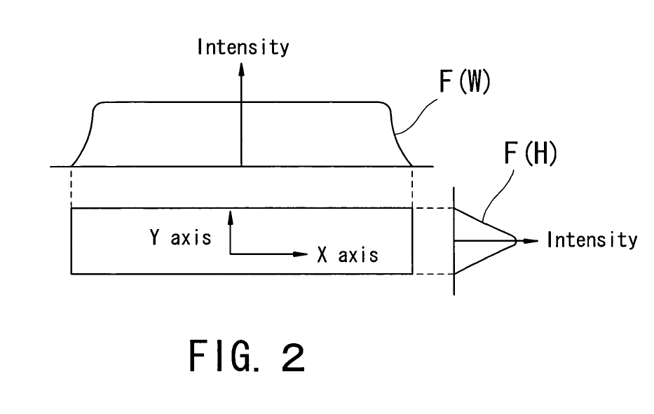

Next, an example will be described where the present invention is applied to an ablation apparatus for corneal surgery. FIG. 1 is a view showing a schematic configuration of an irradiation optical system and a control system in the ablation apparatus for corneal surgery. The present embodiment employs a laser light source 1 emitting a pulsed excimer laser beam with a wavelength of 193 nm. As shown in FIG. 2, a typical cross-sectional shape of the laser beam orthogonal to an irradiation optical axis L1 is a narrow rectangle. Also, intensity distribution (energy distribution) of the laser beam shows approximately uniform distribution F(W) in a longitudinal direction of the cross section (the direction of an x axis) and the Gaussian distribution F(H) in a direction perpendicular to the longitudinal direction (the direction of a y axis). It should be noted that the cross section of the laser beam emitted from the light source 1 may be made to form a desired rectangular shape by beam shaping means such as an expander lens, if necessary.

The laser beam emitted from the light source 1 is reflected and deflected by a plane mirror 2, and it is further reflected and deflected by a plane mirror 3. The mirror 3 is moved by a mirror moving device 4 in a direction of an arrow A along the optical axis L1 to have the laser beam make a parallel movement (scan) in the direction of the Gaussian distribution. Thereby, ablation in uniform depth may be performed (reference should be made to U.S. Pat. No. 5,507,799 corresponding to Japanese Patent Application Unexamined Publication No. Hei4-242644 for details).

An image rotator 5 is rotated on the optical axis L1 by an image rotator driving device 6 so that the laser beam reflected by the mirror 3 is rotated around the optical axis L1 (reference should be made to U.S. Pat. No. 5,637,109 corresponding to Japanese Patent Unexamined Application Publication No. Hei6-114083 for details).

A circular opening region (opening diameter) in a circular aperture plate 7 is changed by a circular aperture plate driving device 8 so as to restrict an ablation zone. Further, a slit opening region (opening width) in a slit aperture plate 9 is changed by a slit aperture plate driving device 10 so as to restrict the ablation zone, and a direction of the slit opening region is also changed as it is rotated on the optical axis L1. A lens 15 (corresponding to the convex lens 207 previously described) projects images of the opening regions in the circular aperture plate 7 and the slit aperture plate 9 onto a cornea Ec of a patient's eye E so as to define the ablation zone.

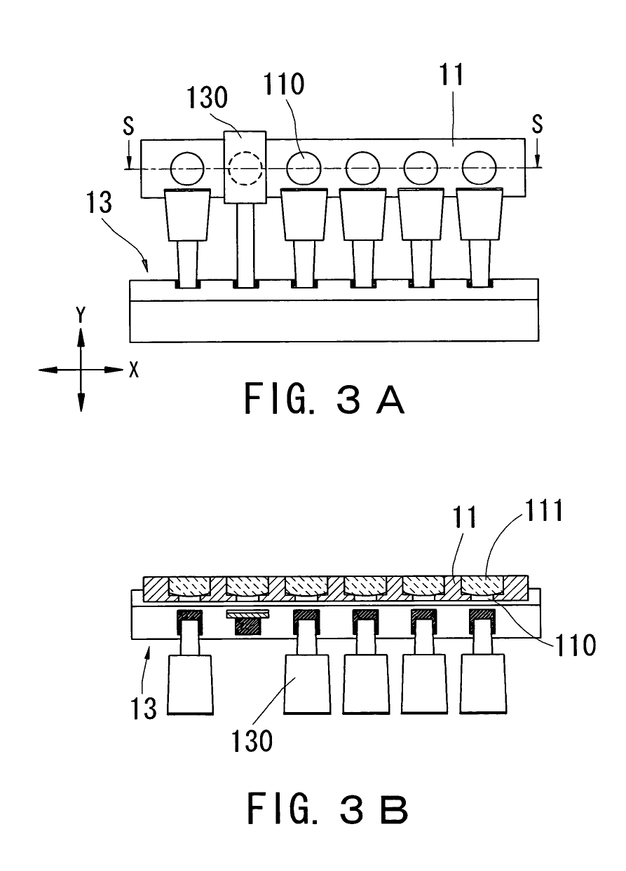

A dividing aperture plate 11 is arranged insertably and removably between the slit aperture plate 9 and the lens 15. The dividing aperture plate 11, as combined with a shutter device 13, further restricts the ablation zone. When the dividing aperture plate 11 is viewed from the side of the cornea Ec, a plurality of small circular openings 110 (six openings in the present embodiment) having approximately the same size and shape are arranged side by side, as shown in FIG. 3A. Each of the small openings 110 corresponds to the small opening of the aperture 205 previously described. In the present embodiment, each of the small openings 110 has a diameter of 3.2 mm. The cross section of the laser beam can be selectively divided and irradiated by selectively covering and uncovering those small openings 110 with each of shutter plates 130 of the shutter device 13.

As shown in FIG. 3B, each of the small openings 110 is provided with an aspherical optical element 111 (corresponding to the aspherical optical element 203 previously described) on the light source 1 side for making the intensity distribution of the laser beam passed through the small opening 110 to have a convex shape. The aspherical optical element 111 is mounted at a position preferably in the vicinity of the small openings 110. The aspherical optical member 111 is made of synthetic fused quartz, and has a flat shape on the laser light source 1 side and an aspherical shape on the cornea Ec side. FIG. 3B is a cross-sectional view of FIG. 3A observed from an S direction.

The dividing aperture plate 11 may be two-dimensionally moved in X and Y directions perpendicular to the optical axis L1 by a dividing aperture plate moving device 12, and the shutter device 13 may be moved in the same directions by a shutter driving/moving device 14. Further, the shutter driving/moving device 14 opens and closes each of the shutter plates 130 by controlling the shutter device 13. It should be noted that the shutter plates 130 may be opened and closed by sliding rather than by rotating as shown in FIGS. 3A and 3B.

A dichroic mirror 16 has a property of reflecting an excimer laser beam having a wavelength of 193 nm and transmitting visible light and infrared light. The laser beam transmitted through the lens 15 is reflected and deflected by the dichroic mirror 16 so as to be guided to and irradiated onto the cornea Ec. An observation optical system 17 having a binocular microscope is disposed above the dichroic mirror 16 (the description of the observation optical system 17 is omitted since it is irrelevant to the present invention). A dichroic mirror 18a has a property of reflecting infrared light and transmitting visible light. Reference numeral 18b is a plane mirror, and an eye position detection optical system 19 detects a position of the patient's eye E (reference should be made to U.S. Pat. No. 6,159,202 corresponding to Japanese Patent Application Unexamined Publication No. Hei9-149914 for details about the eye position detection optical system 19).

A control device 20 controls the entire apparatus including the light source 1, the moving device 4, the driving devices 6, 8, and 10, the moving device 12, the driving/moving device 14, and so on. A data input device 21 is used to input ablation data for the cornea Ec and the like.

Operations in keratorefractive surgery performed by the apparatus having a constitution as above will be described. In the case of removing a rotationally symmetrical spherical component for myopic correction, ablation is performed as follows. The ablation zone is restricted by the circular aperture plate 7, and the mirror 3 is moved in sequence so that the laser beam is moved (scanned) in the direction of the Gaussian distribution. Every time the laser beam finishes moving (scan) in one direction (performing one scan), the image rotator 5 is rotated to change the direction of the laser beam's movement (scan), and the zone restricted by the circular aperture plate 7 may be ablated. By repeating this procedure every time the size of the opening region in the circular aperture plate 7 is changed, the spherical component can be ablated, whereby the central portion of the cornea may be ablated deeply, and the peripheral portion of the cornea may be ablated shallowly. In the case of ablation so as to remove a linearly symmetrical component, the same control is performed using the slit aperture plate 9 instead of the circular aperture plate 7.

Further, in the case of performing partial ablation so as to remove an asymmetric component (irregular astigmatic component), the dividing aperture plate 11 is employed. The dividing aperture plate 11 and the shutter device 13 are placed on an optical path to control positions of the small openings 110 and to selectively cover and uncover the small openings 110 by the shutter plate 130. Thereby, only the laser beam of a small spot passed through the uncovered small opening(s) 110 is irradiated onto the cornea Ec.

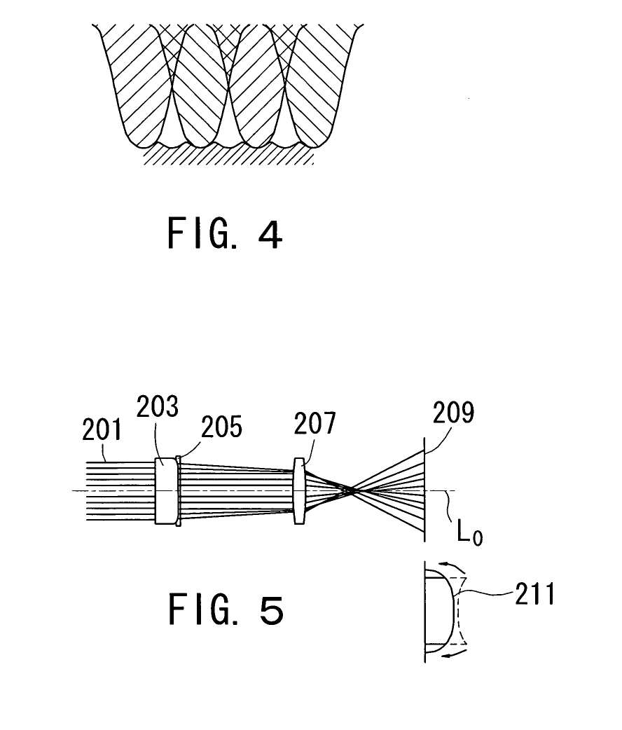

FIG. 4 is a view illustrating irradiation performed by interlocking the laser beam of a small spot. The irradiation of the laser beam having the intensity distribution in a convex shape makes a cross section subject to ablation also in a convex shape. The laser beam as above is interlocked and irradiated at a predetermined ratio to obtain a smooth ablation surface. An ablation amount at each position may be controlled by irradiation time and the number of scan.

In the apparatus in the example given above, the dividing aperture plate 11 is used only at the time of the partial ablation of the aspherical component. However, it may be used also at the time of the ablation of the spherical component and the cylindrical component.

Further, the irradiation position of the laser beam of a small spot passed through the small opening(s) 110 may be moved by moving the lens 15 within a plane intersecting at right angles with the optical axis L1 instead of moving the dividing aperture plate 11. Alternatively, another constitution may be employed where the laser beam transmitted through the lens 15 is scanned by using a galvano-mirror or the like.

Furthermore, the apparatus for ablating corneal tissue has been illustrated hereinbefore; however, the present invention may be applied to an apparatus for ablating eye tissue such as a sclera.

As described above, according to the present invention, even when the laser beam is passed through the small opening of the aperture to be formed into a small spot, the intensity distribution in a convex shape may be obtained at the irradiation surface.

The foregoing description of the preferred embodiments of the invention has been presented for purposes of illustration and description. It is not intended to be exhaustive or to limit the invention to the precise form disclosed, and modifications and variations are possible in the light of the above teachings or may be acquired from practice of the invention. The embodiments chosen and described in order to explain the principles of the invention and its practical application to enable one skilled in the art to utilize the invention in various embodiments and with various modifications as are suited to the particular use contemplated. It is intended that the scope of the invention be defined by the claims appended hereto, and their equivalents.

What is claimed is:

1. An ablation apparatus for ablating an object with a laser beam, the apparatus comprising:
a laser light source for emitting the laser beam which effects ablation to the object;
an irradiation optical system for directing and irradiating the laser beam emitted from the laser light source onto an irradiation surface of the object;
an aperture, arranged in the irradiation optical system, having an opening;
a convex lens, arranged in the irradiation optical system, for once collecting the laser beam passed through the opening of the aperture, and then directing the laser beam onto the irradiation surface at a defocus position; and
an aspherical optical element, arranged in the irradiation optical system, for making intensity distribution of the laser beam after passing through the opening of the aperture to be intensity distribution in a convex shape, wherein an aspherical shape of the aspherical optical element is a curved shape where a radius of curvature at a local surface is reduced toward a peripheral portion from an optical axis.

2. The ablation apparatus according to claim 1, wherein
a focal length f of the convex lens is within a range of 50 to 500 (mm),
the aspherical shape of the aspherical optical element is obtained by a formula Z=−exp[a×Y⁵]+1 giving an exponential function wherein Y indicates a distance (mm) from the optical axis and Z indicates a sag amount (mm), and
an exponential coefficient “a” is within a range of 0.00006×exp[−0.0009×f]≦a≦0.0005×exp[0.0002×f].

3. The ablation apparatus according to claim 2, wherein an opening diameters of the aperture is within a range of 0.4256×a^{-0.185}≦φ≦1.128×a^{-0.1508} (mm).

4. The ablation apparatus according to claim 3, wherein a defocus amount L from a focal point of the convex lens to the irradiation surface is within a range of 0.8×(f/φ)≦L≦2.0×(f/φ) (mm).

5. The ablation apparatus according to claim 2, wherein a distance d from the aspherical optical element to the convex lens is within a range of 3.2448×f−274.51≦d≦4.1520×f−40.647 (mm).

6. The ablation apparatus according to claim 1, wherein the aspherical optical element has a flat shape on a laser light source side and the aspherical shape on an irradiation surface side.

7. The ablation apparatus according to claim 1, wherein the aspherical optical element is arranged in the vicinity of the opening of the aperture.

8. The ablation apparatus according to claim 1, wherein the aspherical optical element makes the intensity distribution of the laser beam after passing through the opening of the aperture to be intensity distribution where intensity in a central portion is relatively high and intensity in the peripheral portion is relatively low.

9. The ablation apparatus according to claim 1, further comprising a moving unit which moves the aperture and the aspherical optical element inside and outside an optical path of the laser beam.

10. The ablation apparatus according to claim 1, wherein the aperture has a fixed opening and profiles a large cross-sectional area of the laser beam into a small area.
